(19) United States
(12) Patent Application Publication
(10) Pub. No.: US 2015/0344843 A1
(43) Pub. Date: Dec. 3, 2015

(54) DETERGENT TREATED BIOLOGICAL TISSUE WITH LOW RESIDUAL DETERGENT AND METHOD OF PRODUCING THE SAME

(71) Applicant: Bacterin International, Inc., Belgrade, MT (US)

(72) Inventor: Mark Schallenberger, Bozeman, MT (US)

(21) Appl. No.: 14/721,932

(22) Filed: May 26, 2015

Related U.S. Application Data
(60) Provisional application No. 62/004,765, filed on May 29, 2014.

(51) Int. Cl. C12N 5/071

(52) U.S. Cl. CPC C12N 5/0625

(57) ABSTRACT

This invention relates to a detergent treated biological tissue with low residual detergent and methods of making the same. The low residual detergent is a clinically beneficial aspect of the biological tissue.

### CROSS REFERENCE TO RELATED APPLICATIONS

This application claims priority and the benefit under 35 U.S.C. §119(e) to U.S. Provisional Patent Application Ser. No. 62/004,765 filed May 29, 2014, which is incorporated herein in its entirety by reference.

### FIELD OF THE INVENTION

This invention relates to a detergent treated biological tissue with low residual detergent and a method of manufacturing thereof. The low residual detergent is a clinically beneficial aspect of the biological tissue.

### BACKGROUND

Biological tissues are commonly used for the surgical reconstruction of a variety of tissue types in humans and other animals. The biological tissue can be surgically implanted, transplanted, or infused into a host recipient using surgical techniques commonly known in the art. In general, the extracellular matrix of biological tissue is recognized in the host recipient as a scaffold for tissue and the host infiltrates the biological tissue with the appropriate progenitor cells for remodeling and integration with host tissue. In many cases, the surgical use of biological tissues augments healing and produces better regenerative outcomes.

It is well known in the art that detergent treatment enhances regenerative capabilities of biological tissues by removing the immunogenic molecules that lead to immunological rejection of these tissues by the host recipient. In some instances, the detergent treatment results in partial or complete decellularization of the biological tissue, which results in a significantly reduced immunological reaction once the biological tissue is implanted into a patient.

Detergents by their nature can be cytotoxic at high concentrations. High levels of residual detergent on biological tissue have been shown to adversely affect clinical outcomes. In some examples, tissue with high levels of detergent acts as an irritant once implanted and results in a negative host response to the biological tissue. Additionally, biological tissue with high residual detergent has been shown to be recellularized and remodeled more slowly than biological tissue with lower residual detergent resulting in delayed regeneration. Specifically, research conducted by Rieder, et. al. [Rieder E, Kasimir M T, Silberhumer G, Seebacher G, Wolner E, Simon P, Weigel G. Decellularization protocols of porcine heart valves differ importantly in efficiency of cell removal and susceptibility of the matrix to recellularization with human vascular cells. J Thorac Cardiovasc Surg. 2004, 127, 399-405] demonstrated that porcine heart valves treated with sodium dodecyl sulfate (SDS) had a toxic influence on endothelial cells and myofibroblasts seeded onto the treated tissue's surface. Similarly, Cartmell and Dunn [Cartmell J S, Dunn M G. Development of cell-seeded patellar tendon allografts for anterior cruciate ligament reconstruction. Tissue Eng. 2004, 10, 1065-1075] have demonstrated that fibroblast proliferation was impeded on SDS treated patellar tendons. Therefore, it is clinically important to produce biological tissue with low residual detergent to allow for the best possible patient outcomes.

While several studies have reported a method of removing detergent from detergent treated biological tissue, example procedures in the literature are time consuming and/or produce tissue with relatively high residual levels of detergent. For example, Gratzer and coworkers have reported two procedures for removing residual detergent from SDS decellularized porcine tendons. The first procedure consisted of soaking the tissue in a pH 9.0 Tris buffer for 24 hours. The second procedure consisted of soaking the tissue in 75% ethanol for 24 hours. In each case the amount of residual detergent was still very high with the pH 9.0 Tris buffer soak producing tissue with 1,380 ng residual SDS/mg tissue and 75% ethanol soak producing tissue with 440 ng residual SDS/mg tissue. [Gratzer P F, Harrison R D, Woods T. Matrix alteration and not residual sodium dodecyl sulfate cytotoxicity affects the cellular repopulation of a decellularized matrix. Tissue Eng. 2006, 12, 2975-2983.] Additionally, U.S. Pat. No. 5,336,616, which is incorporated in its entirety by reference, describes a method for producing SDS decellularized human dermis. To remove residual SDS, the patent teaches that the tissue should be subjected to three, five-minute Hanks balanced salt solution washes. While the amount of residual SDS is not described in the patent, following these instructions yields tissue with residual SDS in excess of 1,000 ng SDS/mg tissue. Therefore, there is a pronounced need in the field for biological tissues with lower residual detergent and improved methods of manufacturing thereof.

### SUMMARY

The invention is directed to a detergent treated biological tissue with a low quantity of residual detergent and a method of making the same.

An aspect of the invention is a detergent treated biological tissue with low levels of residual detergent. The concentration of residual detergent in the detergent treated biological tissue is less than about 200 ng of detergent per mg of tissue.

In some embodiments, the detergent treated biological tissue can be partially decellularized, or completely decellularized. The detergent can be anionic surfactants, cationic surfactants, nonionic surfactants or combinations thereof. In some embodiments, the detergent can be sodium dodecyl sulfate or Triton™ X-100 ((C₂H₄O)ₙC₁₄H₂₂O). The biological can be allograft tissue, autograft tissue, xenograft tissue, cortical bone, cancellous bone, demineralized bone, connective tissue, tendon, pericardium, dermis, cornea, dura mater, fascia, heart valve, ligament, capsular graft, cartilage, collagen, nerve, placental tissue, or combinations thereof. In some embodiments, the tissue may be human dermis.

An aspect of the invention is a method of preparing a low residual detergent biological tissue. The method includes exposing the biological tissue to a detergent containing solution to produce a treated tissue, and exposing the treated tissue to an eliminating solution containing an eliminating agent. The eliminating agent removes at least a portion of residual detergent from the treated tissue to produce a low residual detergent biological tissue.

In some embodiments, the low residual detergent biological tissue can be partially decellularized or completely decellularized. The concentration of residual detergent can be less than about 200 ng of detergent per mg of tissue, and the detergent can be anionic surfactants, cationic surfactants, nonionic surfactants or combinations thereof. The detergent can be sodium dodecyl sulfate or Triton™ X-100 ((C₂H₄O)ₙC₁₄H₂₂O), and the concentration of the detergent can be between about 0.01% and 10%. The biological tissue can be exposed to the detergent containing solution for between about 0.01 hours and 100 hours. The eliminating agent can also be a disinfection agent, and can be selected from alcohols, aldehydes, peroxides, amides, phenolic compounds, heterocyclic compounds, quaternary ammonium compounds, iodine derivatives, iodine formulations, and combinations thereof. The eliminating agent can be povidone iodine, a derivative of povidone iodine, formulation of povidone iodine, or combinations thereof. The eliminating agent can be at a concentration of between about 0.01% and 90%. The biological tissue can be exposed to the eliminating solution for between about 0.01 hours and about 100 hours. The biological tissue can be exposed sequentially to a plurality of eliminating solutions each containing an eliminating agent, in some embodiments, the first eliminating first agent can be ethanol and the second agent can be povidone iodine. The biological tissue can be allograft tissue, autograft tissue, and xenograft tissue, cortical bone, cancellous bone, demineralized bone, connective tissue, tendon, pericardium, dermis, cornea, dura mater, fascia, heart valve, ligament, capsular graft, cartilage, collagen, nerve, placental tissue, and combinations thereof. In some embodiments, the biological tissue can be a human soft tissue allograft or human dermis.

### DETAILED DESCRIPTION OF THE INVENTION

The invention is directed to a detergent treated biological tissue with a low quantity of residual detergent. The invention is also directed toward a method of preparing a detergent treated biological tissue with low residual detergent by exposing the tissue to a detergent containing solution then exposing the tissue to an eliminating solution containing an eliminating agent that removes the detergent.

An advantage of the invention is that residual detergent has been shown to negatively affect the clinical performance of biological tissues. As such, a tissue with low residual detergent has a distinct clinical advantage over tissue with high residual detergent. This clinical advantage may be observed through faster tissue recellularization, faster tissue remodeling with host tissue, faster wound regeneration and repair, or through lower inflammation and irritation of the host tissue surrounding the biological tissue following implantation.

“Detergent” as used herein, broadly includes, but is not limited to, anionic surfactants, cationic surfactants, nonionic surfactants or combinations thereof. Examples of these types of detergents broadly include, but are not limited to, ammonium lauryl sulfate, dioctyl sodium sulfosuccinate, perfluorobutanesulfonic acid, perfluorononanoic acid, perfluorooctanesulfonic acid, perfluorooctanoic acid, potassium lauryl sulfate, sodium dodecyl sulfate, sodium dodecylbenzenesulfonate, sodium laureth sulfate, sodium lauroyl sarcosinate, sodium myreth sulfate, sodium pareth sulfate, sodium stearate, benzalkonium chloride, benzethonium chloride, bronidox, cetrimonium bromide, cetrimonium chloride, dimethyldioctadecylammonium chloride, lauryl methyl gluceth-10 hydroxypropyl dimonium chloride, tetramethylammonium hydroxide, cetomacrogol 1000 (alkyl polyglycol ether), cetostearyl alcohol, cetyl alcohol, cocamide DEA, cocamide MEA, cecyl glucoside, isoceteth-20 (polyethylene glycol ether of isocetyl alcohol), lauryl glucoside, monolaurin, nonidet P-40 (Alpha-(para-Nonylphenyl)-omegahydroxynona-(oxyethylene)), nonoxynol-9 (nonylphenyl polyethylene glycol ether), nonoxynols, octaethylene glycol monododecyl ether, octyl glucoside, oleyl alcohol, pentaethylene glycol monododecyl ether, poloxamer, poloxamer 407 (poloxamers), polyglycerol polyricinoleate, polysorbate, polysorbate 20 (polyoxyethylene derivative), polysorbate 80 (Polyethylene glycol sorbitan monooleate), sorbitan monostearate, sorbitan tristearate, stearyl alcohol, Triton™ X-100 ((C₂H₄O)ₙC₁₄H₂₂O), TWEEN® 80 (polyethylene glycol sorbitan monooleate) and combinations thereof.

In some embodiments, the detergent used in this invention may be elected from the group of alkyl sulfates. In some embodiments, the alkyl sulfate may be selected from the group of salts of dodecyl sulfate. In some embodiments, the salt of dodecyl sulfate may be sodium dodecyl sulfate (SDS). The use of SDS in biological tissue processing is commonly known in the art and multiple commercially available products currently employ SDS in the processing of the biological tissue.

“Biological tissue” as used herein, broadly includes, but is not limited to, allograft tissue, autograft tissue and xenograft tissue. “Allograft tissue” is defined as a tissue derived from a non-identical donor of the same species. “Autograft tissue” is defined as tissue derived from and implanted into the same identical patient. “Xenograft tissue” is defined as tissue derived from a non-identical donor of a different species. Examples of these types of biological tissues broadly include, but not limited to, cortical bone, cancellous bone, demineralized bone, connective tissue, tendon, pericardium, dermis, cornea, dura matter, fascia, heart valve, ligament, capsular graft, cartilage, collagen, nerve, placental tissue, and combinations thereof. In some embodiments, the biological tissue used in this invention may be selected from the group of human allografts. In some embodiments the human allograft may be human dermis.

In one aspect of the invention, the detergent treated biological tissue has low levels of residual detergent. In some embodiments of the invention, the detergent treatment results in partial or complete decellularization of the biological tissue. The low residual level remaining in the tissue following treatment can be between about 0.05 ng of detergent/mg of tissue to about 200 ng or detergent/mg of tissue. In some embodiments, the low residual level may be less than about 200 ng of detergent/mg of tissue. In some embodiments, the low residual level may be less than about 100 ng of detergent/mg of tissue. In some embodiments, the low residual level may be less than about 50 ng of detergent/mg of tissue.

In another aspect of the invention a method is presented to prepare a detergent treated biological tissue with low residual detergent by exposing the tissue to a detergent containing solution and then exposing the tissue to an eliminating solution containing an eliminating agent that removes the detergent. In some embodiments the detergent treatment results in partial or complete decellularization of the biological tissue. In some embodiments, the method produces biological tissue with less than about 200 ng of detergent/mg of tissue. In some embodiments, the method produces biological tissue with less than about 100 ng of detergent/mg of tissue. In some embodiments, the method produces biological tissue with less than about 50 ng of detergent/mg of tissue. In some embodiments, the detergent concentration in an existing tissue sample may be reduced by exposing the tissue to an eliminating solution containing an agent that removes the detergent.

In some embodiments, the tissue may be dried after it has been exposed to the detergent, but before exposure to the eliminating solution. The tissue may be dried at a temperature between about room temperature and about 100° C. The tissue may be dried for between about 5 minutes to about 48 hours.

The detergent containing solution may comprise the detergent and a solvent. The solvent may be an aqueous solvent selected from the group consisting of water (tap, distilled, deionized, or the like), normal saline, phosphate buffered saline, Hank's balanced salt solution, other buffers and combinations thereof. In some embodiments, the solvent may be an alcohol-based solution. The alcohol of the alcohol-based solution may be any suitable alcohol, including but not limited to, methanol, ethanol, isopropanol, other alcohols, or combinations thereof. In some embodiments, the solvent may consist of a combination of aqueous and alcohol-based solvents.

Methods of detergent decellularization of biological tissue are well known in the art. In some embodiments of this invention, the biological tissue may be exposed to a solution of an anionic detergent. In some embodiments, the anionic detergent may be sodium dodecyl sulfate. In some embodiments, the concentration of the solution may be between about 0.01% and about 10%, or between about 0.1% and about 1%. In some embodiments, the concentration of the solution may be about 0.5%. In some embodiments the biological tissue may be exposed to the solution for between about 0.01 and about 100 hours, between about 0.1 hours and about 10 hours, or between about 0.5 hours and about 5 hours. In some embodiments, the biological tissue may be exposed to the solution for about 1 hour.

In some embodiments of the invention, the method of removing the residual detergent involves exposing the detergent treated biological tissue to an eliminating solution that contains an eliminating agent that removes the detergent. In some embodiments, the eliminating agent may have complementary electrostatic or physical interactions with detergent. In some embodiments, the eliminating agent may form a chemical bond with the detergent.

In some embodiments, the eliminating agent may also be a disinfecting agent. This provides the specific advantage of reducing the bioburden on and within the biological tissue while simultaneously removing the residual detergent from the biological tissue. In some embodiments, the disinfecting agent may be selected from the group of alcohols, aldehydes, peroxides, amides, phenolic compounds, heterocyclic compounds, quaternary ammonium compounds, iodine derivatives, iodine formulations, and combinations thereof. In some embodiments, the disinfecting agent may be povidone iodine (PVP-I).

In some embodiments, the eliminating agent may contain a polymer that binds to surfactants including, but not limited to, polyvinylpyrrolidone, poly(diallyldimethylammonium chloride, methoxyhexa(ethylene glycol) methacrylate, 2-(dimethylamino)ethyl methacrylate, and combinations thereof.

In some embodiments, the concentration of the eliminating agent in the eliminating solution may be between about 0.01% and about 90%, or between about 0.1% and about 50%. In some embodiments, the concentration of the eliminating agent in the eliminating solution may be about 5%. In some embodiments, the biological tissue may be exposed to the eliminating solution for between about 0.01 to about 100 hours, between about 0.1 hours to about 20 hours, or between about 0.5 hours and about 10 hours. In some embodiments, the biological tissue may be exposed to the eliminating solution for about 0.5 hours.

In some embodiments, the detergent treated biological tissue may be exposed to a plurality of eliminating solutions. The plurality of eliminating solutions may each contain a different eliminating agent, though it is understood that the same agent could be used multiple times. In some embodiments, at least some of the plurality of eliminating solutions may contain the same eliminating agent, though the eliminating solution may contain different solvents. In some embodiments, one of the eliminating agents is PVP-I and the other is ethanol.

The eliminating solution may be an aqueous solution, wherein the aqueous base may be at least one of water, normal saline, phosphate buffered saline, Hank's balanced salt solution, other buffers and combinations thereof. In some embodiments, the eliminating solution may be an alcohol-based solution. The alcohol may be selected from the group consisting of methanol, ethanol, isopropanol, or other alcohols and combinations thereof. In some embodiments, the eliminating solution may be a combination of aqueous and alcohol-based solutions.

Following subjection of the tissue to a detergent solution and any detergent eliminating solution, the tissue may be preserved using a variety of methods well known in the art. Examples include but not limited to freeze drying, freezing, cryopreservation, and sterilization.

### EXAMPLES

### Example 1

### Cytotoxic Level of SDS

To determine the cytotoxic threshold of SDS, an MEM endpoint dilution assay for liquids using L-929 mouse fibroblast cells was performed. Briefly, solutions of Eagle's minimum essential medium with about 5% fetal bovine serum were created with various concentrations of SDS. One mL of the SDS containing medium was placed in a disposable tissue culture labware and a monoloyer of L-929 mouse fibroblasts was placed on top of the media. The cells were incubated for about 24 hours at about 37° C. in a humidified atmosphere of about 5% CO₂ in air. The mouse fibroblasts were then macroscopically examined using the semi-quantitative scale defined in Table 1.

TABLE 1

| Score | Pass/Fail | Definition |
|---|---|---|
| 0 | Pass | Discrete intracytoplasmic granules, no cell lysis, |
| | | no reduction of cell growth |
| 1 | Pass | Not more than 20% of the cells are round, |
| | | loosely attached, and without |
| | | intracytoplasmic granules, or show changes in |
| | | morphology; occasional lysed |
| | | cells are present; only slight growth |
| | | inhibition observable |
| 2 | Pass | Not more than 50% of the cells are round, |
| | | devoid of intracytoplasmic |
| | | granules, no extensive cell lysis; not more |
| | | than 50% growth inhibition |
| | | observable |
| 3 | Fail | Not more than 70% of the cell layers contain |
| | | rounded cells or are lysed; cell |
| | | layers not completely destroyed, but more |
| | | than 50% growth inhibition |
| | | observable |
| 4 | Fail | Nearly complete or complete destruction |
| | | of the cell layers |

Appropriate positive and negative controls were assessed to ensure the validity of the assay. The results of the testing are described in Table 2.

TABLE 2

| | SDS Concentration (μg/mL) | | | | | | | | |
|---|---|---|---|---|---|---|---|---|---|
| | 32 | 29 | 26 | 23 | 20 | 17 | 16 | 8 | 4 |
| Cytotoxicity Score | 4 | 4 | 4 | 1 | 0 | 0 | 0 | 0 | 0 |
| Pass/Fail | Fail | Fail | Fail | Pass | Pass | Pass | Pass | Pass | Pass |

The SDS concentrations in Table 2 are approximate values. The testing demonstrated that the limit of toxicity for SDS is between about 26 μg/mL and about 23 μg/mL. This corresponds to about 86-98 ng of SDS/mg of tissue. As such for tissue to be non-cytotoxic, it should contain less than about 86 ng of SDS/mg of tissue.

To confirm this non-cytotoxic concentration threshold in vivo, samples of acellular human dermis that contained approximately 90 ng of SDS/mg of tissue were evaluated in a two and four week intramuscular implantation test in rabbits. Pieces of acellular dermis were cut into about 1 mm×1 mm×1 cm samples and implanted into the paravertebral muscles of New Zealand White rabbits. The incision site was closed with non-absorbable sutures and the animals were monitored daily throughout the in-life portion of the study. Following two or four weeks, rabbits were humanely euthanized and the implant sites were harvested and fixed in about 10% neutral buffered formalin. The samples were then processed into paraffin, cut, and stained for light microscopy. The slides were interpreted by a qualified pathologist. The SDS containing samples performed as well as a control sample with no SDS in terms of cellular integration, inflammation, and tissue remodeling. This confirms the cytotoxic threshold determined through the in vitro work.

### Example 2

### Assay for Quantifying SDS in Tissue

To determine the quantity of residual SDS in SDS decellularized tissue an assay was adopted from Hayashi, K. A rapid determination of sodium dodecyl sulfate with methylene blue. Anal Biochem. (1975); 67:503-506. Briefly, approximately about 1 cm×about 1 cm pieces of tissue were weighed and placed in an about 15 mL culture tube. About six milliliters of water were added to the culture tube and the sample was sonicated at about 55° C. for about 2.5 hours to liberate all of the residual SDS tightly bound to the tissue. The quantity of SDS in the solution was then quantified by mixing about 1.5 mL of the sonication solution (or an appropriate dilution if needed) with about 1.5 mL of a methylene blue reagent (consisting of about 250 mg methylene blue, about 50 g of sodium sulfate, and about 10 mL concentrated sulfuric acid in about 1 L of water) in a glass culture tube. The solution was lightly vortexed and then about 3.0 mL of chloroform was added to the culture tube. The resulting solution was vigorously vortexed and then centrifuged at about 2000 rpm for about 1 minute to separate the aqueous and organic layers. The absorbance at about 651 nm of the organic layer was then measured which is directly proportional to the concentration of SDS in the sonication solution. This value was compared to a standard curve prepared with known concentrations of SDS in water. This concentration was then used to calculate the amount of SDS per mg of tissue.

### Example 3

### Decellularization of Human Dermis

Human donor skin was harvested from cadavers under aseptic conditions and stored under frozen conditions until the start of processing. The skin was thawed and the blood, lipids, and adipose tissue were removed by rinsing in sterile water and using an amalgatome. The epidermis of the skin is then released from the dermis of the skin by soaking in a solution of about 1 M NaCl for at least about 12 hours. Following the about 1 M NaCl soak, any remaining portions of the epidermis were removed using forceps or an osteotome. The skin was then briefly rinsed two times with sterile water to remove the residual NaCl solution. The skin was then decellularized by soaking it in an about 0.5% weight by volume solution of SDS in water. The skin was allowed to soak in the SDS solution for about 1 hour yielding predominately acellular human dermis. The acellular dermis was then briefly rinsed two times with sterile water to remove any residual decellularization solution. Tissue processed using this procedure was determined to possess approximately about 1,800 ng of residual SDS/mg of tissue using the assay described in Example 2.

### Example 4

### Efficacy of Steps to Remove the Residual SDS

Samples of SDS decellularized dermis produced using the procedure described in Example 3 were subjected to various solutions to measure the efficacy of these solutions at removing the residual SDS. The quantity of residual SDS was determined before and after the exposure to the various solutions using the procedure outlined in Example 2 so that a percent reduction could be calculated. Table 3 outlines the efficacy of various solutions.

TABLE 3

| | | | % Reduction |
|---|---|---|---|
| Solution | Time | | of SDS |
| Water | 30 | min | 6.0% |
| 0.9% NaCl in Water | 30 | min | 14.2% |
| 5% PVP-I in Water | 30 | min | 74.7% |
| 25% EtOH in Water | 30 | min | 9.8% |
| 50% EtOH in Water | 30 | min | 37.8% |
| 75% EtOH in Water | 30 | min | 54.4% |

The concentrations, times and % reduction listed in Table 3 are all approximate values. Unexpectedly, the 5% PVP-I soak was shown to be the most effective agent at removing residual SDS, even more so than ethanol which has previously been reported to be an effective SDS removal agent. In addition to removing the residual SDS, the PVP-I soak has the additional benefit of disinfecting the tissue.

### Example 5

### Optimized Process for Removing Residual SDS

From the results generated in Example 4, a full rinse procedure was developed to produce tissue with remarkably little residual SDS. Following the SDS decellularization, the dermis samples were subjected to four, fifteen-minute 75% ethanol soaks, a thirty-minute 5% PVP-I soak, and then three, fifteen-minute saline soaks. The resulting tissue contained about 39 ng of SDS/mg of tissue, a 97.8% reduction of SDS levels following the SDS decellularization in only 135 minutes of treatment. Furthermore, the procedure was shown to produce tissue with a remarkably low level of bioburden (<2.3 colony forming units per piece of tissue as determined using the most probable number method), highlighting the ability of the eliminating solutions to remove residual detergent and disinfect the tissue.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1. A detergent treated biological tissue with low levels of residual detergent wherein the concentration of residual detergent in the detergent treated biological tissue is less than about 200 ng of detergent per mg of tissue.

2. The biological tissue of claim 1, wherein the detergent treated biological tissue is partially decellularized.

3. The biological tissue of claim 1, wherein the detergent treated biological tissue is completely decellularized.

4. The biological tissue of claim 1, wherein the detergent is selected from the group consisting of anionic surfactants, cationic surfactants, nonionic surfactants and combinations thereof.

5. The biological tissue of claim 1, wherein the detergent is sodium dodecyl sulfate.

6. The biological tissue of claim 1, wherein the detergent is Triton™ X-100 ((C₂H₄O)ₙC₁₄H₂₂O).

7. The biological tissue of claim 1, wherein the biological tissue is selected from the group consisting of allograft tissue, autograft tissue and xenograft tissue.

8. The biological tissue of claim 1, wherein the biological tissue is selected from the group consisting of cortical bone, cancellous bone, demineralized bone, connective tissue, tendon, pericardium, dermis, cornea, dura mater, fascia, heart valve, ligament, capsular graft, cartilage, collagen, nerve, placental tissue, and combinations thereof.

9. The biological tissue of claim 1, wherein the biological tissue is human dermis.

10. A method of preparing a low residual detergent biological tissue, comprising:
exposing a biological tissue to a detergent containing solution to produce a treated tissue; and
exposing the treated tissue to an eliminating solution containing an eliminating agent, wherein the eliminating agent in the eliminating solution removes at least a portion of residual detergent from the treated tissue to produce a low residual detergent biological tissue.

11. The method of claim 10, wherein the low residual detergent biological tissue is partially decellularized.

12. The method of claim 10, wherein the low residual detergent biological tissue is completely decellularized.

13. The method of claim 10, wherein a concentration of residual detergent in the low residual detergent biological tissue is less than about 200 ng of detergent per mg of tissue.

14. The method of claim 10, wherein the detergent is selected from the group consisting of anionic surfactants, cationic surfactants, nonionic surfactants and combinations thereof.

15. The method of claim 10, wherein the detergent is sodium dodecyl sulfate.

16. The method of claim 10, wherein the detergent is Triton™ X-100 ((C₂H₄O)ₙC₁₄H₂₂O).

17. The method of claim 10, wherein a concentration of the detergent in the detergent containing solution is between about 0.01% and 10%.

18. The method of claim 10, wherein the biological tissue is exposed to the detergent containing solution for between about 0.01 hours and 100 hours.

19. The method of claim 10, wherein the eliminating agent is also a disinfection agent.

20. The method of claim 10, wherein the eliminating agent is selected from the group consisting of alcohols, aldehydes, peroxides, amides, phenolic compounds, heterocyclic compounds, quaternary ammonium compounds, iodine derivatives, iodine formulations, and combinations thereof.

21. The method of claim 10, wherein the eliminating agent is one of a povidone iodine, a derivative of povidone iodine, or a formulation of povidone iodine.

22. The method of claim 10, wherein a concentration of the eliminating agent in the eliminating solution is between about 0.01% and 90%.

23. The method of claim 10, wherein the biological tissue is exposed to the eliminating solution for between about 0.01 hours and about 100 hours.

24. The method of claim 10, wherein the biological tissue is exposed sequentially to a plurality of eliminating solutions each containing an eliminating agent.

25. The method of claim 24, wherein the first eliminating first agent of the plurality of eliminating solutions is ethanol and the second agent of the plurality of eliminating solutions is povidone iodine.

26. The method of claim 10, wherein the biological tissue is selected from the group consisting of allograft tissue, autograft tissue, and xenograft tissue.

27. The method of claim 10, wherein the biological tissue is selected from the group consisting of a cortical bone, a cancellous bone, a demineralized bone, a connective tissue, a tendon, a pericardium, a dermis, a cornea, a dura mater, a fascia, a heart valve, a ligament, a capsular graft, a cartilage, a collagen, a nerve, a placental tissue, and combinations thereof.

28. The method of claim 10, wherein the biological tissue is a human soft tissue allograft.

29. The method of claim 10, wherein the biological tissue is a human dermis.
